(19) United States
(12) Patent Application Publication
(10) Pub. No.: US 2007/0207228 A1
(43) Pub. Date: Sep. 6, 2007

(54) Composition for Regulating the Trophism of Hair Follicles and the Cutaneous Production of Sebum and Use Thereof in Androgenic Alopecia

(76) Inventors: Giammaria Giuliani, Milano (IT); Anna Benedusi, Pordenone (IT)

Correspondence Address: James V Costigan;Hedman & Costigan, 1185 Avenue of the Americas, New York, NY, 10036-2601, US

(21) Appl. No.: 10/590,543

(22) PCT Filed: Mar. 1, 2005
(86) PCT No.: PCT/IB05/00544

§ 371 Date: Jan. 12, 2007

(30) Foreign Application Priority Data
Mar. 2, 2004 (IT) MI2004A000386
Mar. 2, 2004 (IT) MI2004A000388

(51) Int. Cl. A61K 36/13; A61K 31/7048; A61K 31/353

(52) U.S. Cl. 424/770; 514/27; 514/456

(57) ABSTRACT

The present invention relates to a composition for regulating the trophism of hair follicles and the cutaneous production of sebum and its use in androgenetic alopecia, comprising the synergic association of an extract from Boehmeria Nippononivea with a compound active at the keratin epithelial structure level, selected from specific sulfur-donor compounds and antioxidants. The composition of the invention is suitable for topical and/or oral administration.

The present invention relates to a composition for regulating the trophism of hair follicles and the cutaneous production of sebum and its use in androgenetic alopecia.

In particular, the present invention relates to food supplements and compositions for topical application based on a vegetable extract from a selected plant, which, in combination with other antioxidant active principles, exerts a regulation action on the skin production of sebum and on the trophism of keratin structures, such as hair.

Aesthetical problems relating to seborrheic skin, wherein an excessive production of sebum also causes or accelerates hair loss, have reached a constantly increasing relevance in modern society.

A high percentage of young people have aesthetical problems caused by seborrhea, acne, face furunculosis, excessively greasy hair accompanied by hair thinning. The considerable attention paid to these aesthetic problems by the younger generation and also others, has led to a growing request for products capable of reducing the production of sebum by the sebaceous glands, thus improving an individual's aesthetical appearance.

The cosmetic and pharmaceutical industry has consequently recently developed a wide range of products, mainly directed towards topical use, suitable for treating the excessive production of sebum and hair loss, which is often an unpleasant consequence.

It has been found that a high percentage of the population suffering from seborrhea also has a concomitant problem of hair loss. In the beginning, this was attributed to a suffocation action of the hair bulb due to the excess sebum secreted at the bulb level.

Recent studies have correlated the excessive production of sebum and hair-loss with an increased sensitivity of some skin structures towards an enzyme, 5-α-reductase. In particular, it has been found that the main factor responsible for skin diseases due to the excessive production of sebum, is 5-α-reductase, an enzyme which is mainly expressed specifically at the level of the follicle cells. In particular, it has been observed that this enzyme transforms testosterone, the main male hormone, into its powerful derivative dihydrotestosterone or DHT, one of the main causes of androgenetic alopecia, telogenic effluvium and seborrhea.

Follicles of the scalp areas subject to hair thinning, produce high quantities of this enzyme and, therefore, high quantities of DHT. DHT, in turn, interrupts the normal functions of hair follicles, causing their partial or total destruction.

As it has been observed that a reduced production of DHT prevents further hair loss, at the same time causing new growth in the bald areas or subject to thinning, compounds have been developed which block the activity of 5α-reductase, causing a decrease in the DHT levels.

One of the main substances currently used for oral but also topical administration, in order to block type 2 5-α-reductase, is Finasteride.

Drugs based on Finasteride have obtained favorable success and have proved to be particularly efficient not only in the treatment of alopecia and in promoting hair growth, but are also for preventing further hair thinning and for increasing hair thickness.

The administration of drugs based on Finasteride does, however, cause side-effects, also quite serious ones, such as the reduction of libido, impotence, skin rashes, reduction of the sperm volume, in addition to negatively influencing PSA diagnostic exams. Moreover, this drug has the serious limit of not being administered to women, particularly when pregnant, as its presence in the blood influences the development of the genitals of the fetus.

Another drug, Minoxidil, widely used for the topical treatment of alopecia, has also shown side effects such as migraine, hypertension, eczemas, itch, hot flashes, hypertrichosis and hirsutism.

The necessity is currently felt for products for cosmetic or pharmaceutical use which are effective in preventing and treating hair loss, together with or without seborrhea, devoid of relevant side-effects.

Similarly, many side-effects have been verified in the treatment of seborrhea and acne, caused by the indiscriminate prescription of drugs such as antibiotics, cortisone-based drugs and derivatives of retinoic acid.

Even if these drugs can cause the regression of acne and, in some cases, the reduction of the skin production of sebum, they can, in fact, have side-effects, at times even serious, such as hepatic diseases, skin infections, skin rashes, appearance of skin spots, etc.

As several studies have demonstrated that in many cases there is a common etiology in the development of seborrhea, acne and hair loss, attempts have been made, for the treatment of these diseases, by administering, also systemically, preparations based on antioxidant compounds, such as vitamin E or selenium.

The individual response to these types of treatment is, however, extremely variable and not always satisfactory.

The Applicant has now found that it is possible to obtain an individual satisfactory response to the above problems by combining a selected active principle of a vegetable origin, with one or more compounds which act at the level of the epithelial structures, in particular the keratin.

One of the main objectives of the present invention is therefore to provide a synergic composition suitable for regulating the skin production of sebum and the trophism of hair follicles, based on an active principle of a natural origin, whose administration is substantially without side-effects.

Another objective of the present invention consists in providing an oral integrator based on a synergic association of active principles, effective in preventing and treating androgenetic alopecia or telogenic effluvium and correlated excessive hair greasiness.

Yet another objective of the present invention consists in providing a composition based on a vegetable extract combined with specific nutrients which are suitable for the treatment of skin diseases characterized by an excessive activation of the sebaceous glands, such as seborrhea and acne vulgaris.

A further and not last objective of the present invention is to prepare a synergetic composition suitable for restoring the physiological trophism of hair follicles which can be used for the treatment of bulb atrophy, such as in telogenic effluvium, and for the treatment of bulb hyper-activation such as in hypertrichosis and hirsutism.

To accomplish these and other objectives which will appear more evident in the following description, a composition is provided, in accordance with a first embodiment of the invention, for regulating the skin production of sebum and/or the trophism of hair follicles, comprising an association of

i) an extract of a vegetable origin which inhibits the 5-α-reductase enzyme,

ii) a compound which acts at the level of the epithelial, in particular the keratin, structure, characterized in that said extract of a vegetable origin i) is an extract of Boehmeria Nippononivea, and said compound which acts at the level of the epithelial keratin structures ii) is selected from sulfur donor compounds, antioxidant compounds and mixtures thereof.

In accordance with an embodiment, the sulfur donor compound is a sulphurated amino acid, methyl sulphonyl methane and/or mixtures thereof. Said sulphurated amino acid is suitably selected from cystine, cysteine or methionine and mixtures thereof.

Suitable antioxidant compounds are selected from phenylpropanoid compounds, flavonoids, isoprenoid derivatives and mixtures thereof.

According to an embodiment, said phenylpropanoids are selected from caffeic acid, hydroxytirosole, chlorogenic acid, Teupolioside, Phenylpropanoids from Ajuga reptans, and mixtures thereof.

According to embodiments of the invention:

said flavonoids are selected from quercetine, kaempferole,

the isoflavones are selected from genisteine and daidzeine,

the flavanoles are preferably catechin,

the flavanones are selected from naringenine and resveratrole and mixtures thereof.

Said isoprenoid derivatives are suitably selected from carotenoids, tocopherols, tocotrienols, saponine and mixtures thereof. In accordance with a preferred embodiment of the invention, said antioxidant compounds are selected from isoprenoid compounds, phenyl propanoid, flavonoids and mixtures thereof.

Suitable oxidant agents can be obtained from emblica (Phyllanthus emblica).

It has been found that the association of the Boehmeria Nippononivea extract with the above-mentioned active principles exerts a synergic effect for the regulation of the trophism of some epithelial structures, with particular reference to the sebaceous glands and hair follicles. In particular, the administration of the composition of the invention causes a reduction in sebum secretion with beneficial effects on acne and seborrhea and a regulation of physiological hair growth, with positive results on androgenetic alopecia, telogenic effluvium, hypertrichosis and hirsutism.

The extract from Boehmeria Nippononivea used within the scope of the invention can typically be alcoholic, hydro-alcoholic, glycerin, acetonic, the use of the hydro-alcoholic or acetonic extract being preferred.

It has been found, in fact, that with these two types of extracts it is possible to obtain a final product particularly rich in vegetable substances active in the selective inhibition of the 5-α-reductase enzyme, highly expressed at a follicle level. In addition to this inhibition effect, accompanied by a reduction in the circulating DHT, there is also the effect of the stimulation and protection of the epithelial structures expressed by the antioxidant or sulfur donor components of the invention.

The Boehmeria Nippononivea extract can be advantageously prepared through one of the extraction processes described hereunder.

The preparation of the acetonic extract comprises the following phases:

Grinding of the aerial parts of Boehmeria Nippononivea with a solvent quantity in a ratio 1:10 and 1:30 with the weight of the drug to be extracted

separation of the solid from the liquid and washing of the residue with an additional amount of solvent

concentration and evaporation until the extract is dried.

The preparation of the hydro-alcoholic extract comprises the following phases:

fine grinding of the leaves and aerial parts of Boehmeria Nippononivea

determination of the water content and addition of ethyl alcohol so as to have a drug/solvent ratio equal to about 1:10 by weight.

extraction, repeated two or three times until exhaustion of the material to be extracted

filtering and concentration of the extract by means of solvent evaporation.

possible drying of the extract.

According to another embodiment, the hydro-alcoholic extract from Boehmeria Nippononivea is used as an inhibitor of 5-α-reductase. The hydro-alcoholic extract is particularly active notwithstanding its low concentration of polyunsaturated fatty acids, conveniently lower than 8% and advantageously ranging from 2 to 6% by weight. Typically, the hydro-alcoholic extract has a concentration of polyunsaturated fatty acids ranging from 3.5 to 4.5% by weight. It has thus been observed that the inhibition activity on 5-α-reductase can also be related to the component having a lower lipophilic property, not yet characterized. This effect is surprising as, in the past, the enzyme inhibition action essentially referred to the lipophilic “fatty” component based on polyunsaturated acids.

According to this last embodiment, the extraction of the useful fractions having an inhibitory activity on 5-α-reductase, is done using an alcoholic or hydroalcoholic solution having an alcohol degree ranging from 100 to 950 by volume.

Optimal results in the preparation of vegetable active fractions are obtained using the leaf apparatus of Boehmeria Nippononivea.

A typical preparation of the hydro-alcoholic extract for the uses of the invention comprises the following phases:

fine grinding of the leaves and/or aerial parts of Boehmeria Nippononivea,

determination of the water content and addition of ethyl alcohol so as to have a drug/solvent ratio by weight equal to about 1:10.

extraction, repeated two or three times until exhaustion of the material to be extracted

filtering and concentration of the extract by means of evaporation of the solvent

drying of the extract.

The vegetable extract is usefully obtained by means of a method which includes the following phases:

cleaning the drug (leaves and possibly aerial parts)

drying

grinding, possibly cryogenic grinding

extraction, suitably performed in an appropriate percolator, preferably using food-grade alcohol

clarification by means of centrifugation

liquid concentration

eventual refining by means of chromatography

liquid concentration and

optionally drying, in the case of the preparation of the dry extract.

The extraction phase of the active substances of Boehmeria Nippononivea according to this embodiment is performed by preferably using an amount of hydroalcoholic solvent in a ratio 1:10 and 1:30, with respect to the weight of the drug to be extracted. After the first extraction, there is advantageously a separation of the solid part, or a soaked vegetable part, from the liquid component extracted and a subsequent washing of the residue obtained with an additional quantity of solvent.

The extract rich in vegetable fractions is subsequently concentrated, for example by heating to a temperature conveniently within the range of 20-70° C.

In accordance with this embodiment, the active fractions are advantageously extracted by the addition of a hydro-alcoholic solution in a quantity suitable to obtain a vegetable substance/solvent by weight ratio ranging from 0.5:10 to 2:10 w/v.

The concentrated extract can be used as such, or it can be concentrated by evaporation to dryness.

The synergic composition of the invention can be used both in topical and systemic application, and has proved to be effective in preventing and/or treating affections caused by the activity of 5-α-reductase, for instance the affections caused by an excessive production of sebum such as acne, seborrhea, furunculosis, and affections such as androgenetic alopecia, telogenic effluvium, hair thinning and also hypertrichosis and/or hirsutism.

The composition of the invention has proved to be particularly suitable for the treatment of androgenetic alopecia.

The compositions for topical application of the invention can be either in liquid form such as lotions, solutions or in semi-solid form such as pastes, gels, creams, ointments, masks, transdermic patches with controlled release.

The compositions for local application of the invention can conveniently comprise additives commonly used in cosmetic or pharmaceutical preparations for local use, such as preservatives, antibacterial agents, stabilizers, emulsifying agents, buffers, dyes and other excipients commonly used in cosmetic/pharmaceutical preparation techniques.

In the case of liquid formulations, the synergic active principles of the invention can be conveniently dissolved in a cosmetically/pharmaceutically acceptable liquid medium such as water, alcohol, hydro-alcoholic or glycerin solution, and other media suitable for local application.

For illustrative purposes, the compositions of the invention in liquid form are prepared by dissolving the hydro-soluble vegetable fractions extracted in water and the remaining fractions in alcohol, subsequently joining the different fractions under stirring. The resulting mixture can then be buffered to reach a pH range conveniently selected from 5 to 7 so as to be compatible with the pH of the skin and then filtered and packaged in suitable containers such as bottles or ampoules.

The composition for topical use of the invention is used for application, in an effective quantity, directly on the affected body region to be treated.

For example, in the treatment of androgenetic alopecia, a lotion based on the active principles of the invention is applied directly on the scalp once or more than once a day conveniently for cycles of 2-3 months alternating with rest periods.

Analogously, a composition in the form of a cream can be applied once or more than once a day on the face of a subject affected, for example, by seborrhea or acne, until the remission of the disease.

In the case of a solid or semi-solid formulation, the synergic active principles of the invention are dispersed in cosmetically/pharmaceutically acceptable carriers, commonly used for local application.

The application of the composition of the invention in the form of a cream causes a reduction in the secretion of sebum by the sebaceous glands which is visible after a few days of treatment as a reduction in the oiliness of the body surface treated.

The compositions of the invention for systemic use can be produced in the form of tablets, pills, capsules, solution, suspension, syrup, and in solid forms suitable for the controlled release of the active principles.

Preparation for oral administration of the invention is done according to the common preparation techniques of dietetic and/or pharmaceutical products, by adding one or more physiologically acceptable excipients to the synergic active principles. Physiologically acceptable excipients are therefore used in a blend with suitable preservatives, stabilizers, diluents, carriers and flavoring agents.

For example, a typical composition for oral use is in the form of a tablet with a core containing the active principles described above, inside a coating film. Typically, the coating comprises one or more substances selected from hydroxypropylmethylcellulose, microcrystalline cellulose, stearic acid and suitable dyes such as titanium dioxide, iron oxide (yellow and/or red-E 172) and others.

In the composition of the invention, the synergic active principles of the invention are typically present in varying quantities, normally ranging from 0.001% by weight to 10% by weight, more preferably from 0.1 to 5% by weight.

According to another aspect of the invention, a cosmetic treatment method is provided, which comprises the local application, at the level of the scalp or face, of an effective quantity of a synergic composition described above.

According to another embodiment, a method is provided for regulating the skin production of sebum and the nourishment of hair follicles comprising the administration of a food supplement of the type described above to a subject in need of treatment.

The following examples are provided purely to illustrate the present invention and should in no way be considered as limiting its protection scope as specified by the enclosed claims.

### EXAMPLES

### Example 1

Systemic Use

Integrator based on Boehmeria and isoprenoid-derivative anti-oxidants (carotenoids, tocopherols, tocotrienols, saponine):

Integrator in tablet form suitable for reducing the damage of the keratin structures cause by the oxidative stress indices by sun-rays.

Each tablet contains:

| | | |
|---|---|---|
| Spermidine trihydrochloride | 0.50 | mg |
| Calcium pantothenate | 9 | mg |
| d-Biotin | 0.150 | mg |
| Boehmeria Nippononivea extract | 100 | mg |
| Ajuga reptans | 5 | mg |
| Beta carotene | 7.2 | mg |
| Ubidecarenone | 10.0 | mg |
| Zinc amino acid chelate | 7.5 | mg |
| Copper amino acid chelate | 1.20 | mg |
| Folic acid | 0.30 | mg |
| Microcrystalline cellulose | 17.0 | mg |
| Calcium phosphate bibasic dihydrate | 62.0 | mg |
| Hydroxypropylmethylcellulose | 80.0 | mg |
| Magnesium stearate | 7.90 | mg |
| Silicon dioxide | 1.70 | mg |

### Example 2

Food supplement based on Boehmeria and sulfur donor compounds (sulphurated amino acids, methylsulphonyl methane) in tablet form, suitable for reducing food intake deficiency:

Each table contains:

| | | |
|---|---|---|
| Methionine | 300 | mg |
| Spermidine trihydrochloride | 0.50 | mg |
| Calcium pantothenate | 9 | mg |
| d-Biotin | 0.150 | mg |
| Boehmeria Nippononivea extract | 200 | mg |
| Ajuga reptans | 5 | mg |
| Zinc amino acid chelate | 7.5 | mg |
| Copper amino acid chelate | 1.20 | mg |
| Manganese amino acid chelate | 2.25 | mg |
| Vitamin B6 | 3.0 | mg |
| Folic acid | 0.30 | mg |
| Microcrystalline cellulose | 17.0 | mg |
| Calcium phosphate bibasic dihydrate | 62.0 | mg |
| Hydroxypropylmethylcellulose | 80.0 | mg |
| Magnesium stearate | 7.90 | mg |
| Silicon dioxide | 1.70 | mg |

### Example 3

Food supplement based on Boehmeria and antioxidants of the group of phenylpropanoids (caffeic acid, hydroxytyrosol, chlorogenic acid, ajuga) in tablet form with an anti-aging function.

Each tablet contains:

| | | |
|---|---|---|
| Spermidine trihydrochloride | 0.50 | mg |
| Calcium pantothenate | 9 | mg |
| d-Biotin | 0.150 | mg |
| Boehmeria Nippononivea extract | 100 | mg |
| Ajuga reptans | 5 | mg |
| Zinc amino acid chelate | 7.5 | mg |
| Copper amino acid chelate | 1.20 | mg |
| Folic acid | 0.30 | mg |
| Microcrystalline cellulose | 17.0 | mg |
| Calcium phosphate bibasic dihydrate | 62.0 | mg |
| Hydroxypropylmethylcellulose | 80.0 | mg |
| Magnesium stearate | 7.90 | mg |
| Silicon dioxide | 1.70 | mg |

### Example 4

Food supplement based on Boehmeria and flavonoids (the group of flavonoids comprises: flavonols, quercetin and Kaempferol, —isoflavones, genistein and daidzein—flavanols, catechine, —flavanones, naringenine and resveratrol) in tablet form. The Food supplement is particularly suitable for androgenetic alopecia and telogenic effluvium in women close to the menopause or during menopause.

Each tablet contains:

| | | |
|---|---|---|
| Spermidine trihydrochloride | 0.50 | mg |
| Calcium pantothenate | 9 | mg |
| d-Biotin | 0.150 | mg |
| Soybean isoflavones | 40 | mg |
| (genistein and daidzein) | | |
| Boehmeria Nippononivea extract | 100 | mg |
| Resveratrol | 0.05 | mg |
| Zinc amino acid chelate | 7.5 | mg |
| Copper amino acid chelate | 1.20 | mg |
| Folic acid | 0.30 | mg |
| Microcrystalline cellulose | 17.0 | mg |
| Calcium phosphate bibasic dihydrate | 62.0 | mg |
| Hydroxypropylmethylcellulose | 80.0 | mg |
| Magnesium stearate | 7.90 | mg |
| Silicon dioxide | 1.70 | mg |

### Example 5

Food supplement based on Boehmeria, emblica (Phyllanthus emblica), resveratrol (antioxidants) and soybean isoflavones.

The food supplement, in the form of coated tablets, is particularly suitable for androgenetic alopecia and telogenic effluvium in women close to the menopause or during the menopause.

Each coated tablet contains:

| Nucleus | | |
|---|---|---|
| Boehmeria Nippononivea, | 200 | mg |
| Hydro-alcoholic dry extract | | |
| Emblica dry extract | 100 | mg |
| Soybean isoflavones | 40 | mg |
| Calcium d-Pantothenate | 9 | mg |
| Zinc (as amino acid chelate) | 7.5 | mg |
| Copper (as amino acid chelate) | 1.2 | mg |
| Spermidine trihydrochloride | 0.50 | mg |
| Folic acid | 0.30 | mg |
| d-Biotin | 0.15 | mg |
| Resveratrol | 0.05 | mg |
| Hydroxypropylmethylcellulose | 135 | mg |
| Calcium phosphate bibasic dihydrate | 58 | mg |
| K-carrageenan | 49 | mg |
| Magnesium stearate | 7.005 | mg |
| Silicon dioxide | 5 | mg |
| Coating | | |
| Yellow Iron oxide (E 172) | 0.3 | mg |
| Red Iron oxide (E 172) | 0.2 | mg |
| Hydroxypropylmethyl cellulose | 21.3 | mg |
| Microcrystalline cellulose | 3.2 | mg |
| Stearic acid | 3.2 | mg |
| Titanium dioxide | 5 | mg |

### Example 6

Food supplement in tablet-form suitable for the prevention of androgenetic alopecia in males and females.

Each tablet contains:

| | | |
|---|---|---|
| Boehmeria Nippononivea, | 200 | mg |
| Hydro-alcoholic dry extract | | |
| Taurine | 200 | mg |
| Hydroxypropylmethylcellulose | 110 | mg |
| Calcium phosphate bibasic dihydrate | 46 | mg |
| Microcrystalline cellulose | 46 | mg |
| K-carrageenan | 35 | mg |
| Calcium Pantothenate | 9 | mg |
| Zinc (as amino acid chelate) | 7.5 | mg |
| Magnesium stearate | 7 | mg |
| Dry Ajuga extract | 5 | mg |
| Silicon dioxide | 5 | mg |
| Copper (as amino acid chelate) | 1.20 | mg |
| Quercetin | 0.9 | mg |
| Spermidine trihydrochloride | 0.50 | mg |
| d-Biotin | 0.15 | mg |

### Example 7

Food supplement in tablet form suitable for the prevention of androgenetic alopecia in males and females

Each tablet contains:

| | | |
|---|---|---|
| Spermidine trihydrochloride | 0.50 | mg |
| Calcium pantothenate | 9 | mg |
| d-Biotin | 0.150 | mg |
| Boehmeria Nippononivea extract | 150 | mg |
| Quercetin | 0.90 | mg |
| Taurine | 100 | mg |
| Zinc amino acid chelate | 7.5 | mg |
| Copper amino acid chelate | 1.20 | mg |
| Folic acid | 0.30 | mg |
| Microcrystalline cellulose | 90.0 | mg |
| Calcium phosphate bibasic dihydrate | 80.0 | mg |
| Hydroxypropylmethylcellulose | 52.5 | mg |
| Magnesium stearate | 7.90 | mg |
| Silicon dioxide | 1.70 | mg |

### Example 8

Composition for topical use based on Boehmeria and isoprenoid-derivative antioxidants (carotenoids, tocopherols, tocotrienols, saponine):

Dermatological cream for reducing the hair bulbs and skin damage of UV-ray exposure

The composition comprises:

| | | |
|---|---|---|
| Boehmeria Nippononivea extract | 0.5 | g |
| Spermidine trihydrochloride | 0.50 | mg |
| Calcium pantothenate | 9 | mg |
| d-Biotin | 0.150 | mg |
| Ajuga reptans | 5.0 | mg |
| Macrogol cetosteraryl ether | 5.0 | g |
| Isopropyl myristate | 4.0 | g |
| Propylene glycol | 3.0 | g |
| Glycerin | 3.0 | g |
| White vaseline | 11.0 | g |
| Cetylstearyl alcohol | 9.0 | g |
| Methylene para-oxybenzoate | 0.2 | g |
| Propyl para-oxybenzoate | 0.02 | g |
| Tetrasodium EDTA | 0.1 | g |
| Water | 64.18 | g |

### Example 9

Composition for topical use, useful in telogenic effluvium in males and females, based on Boehmeria and flavonoids:

| | | |
|---|---|---|
| Spermidine trihydrochloride | 2.0 | mg |
| Calcium pantothenate | 30.0 | mg |
| d-Biotin | 0.30 | mg |
| Boehmeria Nippononivea extract | 100 | mg |
| Ajuga reptans | 5.0 | mg |
| Beta glucan | 0.50 | mg |
| Phytotocotrienols | 20 | mg |
| Grapefruit seed extract | 30.0 | mg |
| Disodium EDTA | 3.0 | mg |
| Cremophor | 30 | mg |
| Perfume | 6.0 | mg |
| Citric acid | 1.5 | mg |
| Denatured alcohol | 350 | mg |
| Water | as required to 10 | mL |

### Example 10

Composition for topical use based on Boehmeria and antioxidants of the phenylpropanoid group, particularly suitable for anti-inflammatory action in cases of acne and seborrhea:

| | | |
|---|---|---|
| Boehmeria Nippononivea extract | 0.5 | g |
| Spermidine trihydrochloride | 2.0 | mg |
| Calcium pantothenate | 30.0 | mg |
| d-Biotin | 0.30 | mg |
| Ajuga reptans | 5.0 | mg |
| Emulpharma XL | 5.0 | g |
| Labrafac CC | 5.0 | g |
| White Vaseline | 2.0 | g |
| MOD | 3.0 | g |
| Cetylstearyl alcohol | 2.0 | g |
| Perfume | 0.20 | g |
| Conc. Tocopherol | 0.05 | g |
| Euxil K300 | 0.6 | g |
| Cyclometicone | 0.05 | g |
| Propylene glycol | 3.45 | g |
| Glycerin | 3.2 | g |
| Ultrez 21 | 0.60 | g |
| Tetrasodium EDTA | 0.10 | g |
| AMP | 0.45 | g |
| Water | 73.35 | g |

### Example 11

Composition for local application in the form of an extemporary mask useful in cases of hypertrichosis

| | | |
|---|---|---|
| Boehmeria Nippononivea extract | 8 | g |
| Ajuga reptans | 5.0 | mg |
| Spermidine trihydrochloride | 2.0 | mg |
| Calcium pantothenate | 30.0 | mg |
| d-Biotin | 0.30 | mg |
| Isagel FM alginate | 92 | g |

### Example 12

Composition for topical use based on Boehmeria and isoprenoid antioxidants:

| | | |
|---|---|---|
| Boehmeria Nippononivea extract | 0.5 | g |
| Spermidine trihydrochloride | 2.0 | mg |
| Calcium pantothenate | 30.0 | mg |

| | |
|---|---|
| Water | 58.730% |
| Denatured alcohol | 20.00% |
| Disodium EDTA | 0.050% |
| Glycerin | 2.00% |
| Betaine | 0.500% |
| Pronalen | 1.00% |
| Aristoflex | 1.200% |
| Parsol MCX | 5.00% |
| Parsol 1789 | 3.00% |
| Eusolex | 3.00% |
| Lymnantes alba | as required |
| Butyrospermum parkii | as required |
| Trimethylsilylamodimeticone | as required |
| Rosmarinum officinalis | as required |
| Carotene | as required |
| Cylcopentaxyloxane | 3.00% |

### Example 13

For the evaluation of the efficacy of the food supplement based on Boehmeria nippononivea according to Example 4, whose extracts showed an antioxidant and inhibiting activity of the 5-alpha reductase enzyme, a double-blind clinical study was performed on subjects with telogenic effluvium.

Materials and Methods

A double-blind clinical trial was carried out on 30 healthy consenting volunteers of both sexes and aged between 18 and 60 years, affected by telogenic effluvium for at least three months from the enrollment date. The subjects, having homogeneous clinical characteristics, were divided into three groups (A, B, C), each with 10 subjects, according to a previously defined randomized list.

Capsules containing Boehmeria alone, were administered to group A, the food supplement of Example 4 in retard capsules to group B and placebo capsules to group C.

The treatment, which lasted two months, envisaged the assumption of a capsule a day at breakfast time.

The following parameters were evaluated at the times T₀ (basal recruitment), T₁ (60 days, end of treatment), for each subject:

1. general and dermatological examination, in order to ascertain clinically detectable alterations in the general state of health (important for the inclusion or exclusion from the study, possible concomitant pharmacological therapies, etc.) dermatological evaluation for the exact definition of the trichological diagnosis and exclusion of possible concomitant dermatological pathologies unsuitable for inclusion in the clinical study;

2. microscopic evaluation of the hair bulb and stem to determine the percentage of bulbs in anagen and telogen and to measure the diameter of the hair stem;

3. pull test: evaluation of the pulling resistance of the hair stems, subsequently defined according to the following score:

2=very poor or poor pulling resistance

1=sufficient pulling resistance

0=high pulling resistance

4. wash test: amount of hair lost during washing, done twice a week, by counting the number of hairs collected in the basin at the end of the washing (average subjective values for all the subjects for each washing), expressed in numerical terms;

5. haematochemical analysis: to ascertain possible specific deficiencies referred to or non referred to telogenic effluvium in each individual subject. Particularly, it was useful to exclude specific iron and oligo-element deficiencies such as zinc and magnesium, and evaluate the electrophoresis of the haematic proteins to exclude specific forms of hypoproteinemia, and evaluate the possible increase in haematic proteins after administration of the product;

6. evaluation of the possible side-effects attributable to the administration of the capsules containing the three specific preparations.

Results

Microscopic Evaluation of the Hair Stem

The diameter of the hair stem, from T₀ to T₁, increased by 48.2% in group A, by 51-8% in the subjects of group B and 0.9% in group C. The modifications were extremely significant.

Trichogram

Even if this test, taken alone, is not the most important absolute parameter for evaluating the cyclic phase of hair bulbs, it allows a sufficiently accurate quantification of the percentages of the various cyclic phases of hair bulbs.

An analysis of the data on the modifications of the anagen/telogen phases, following treatment with the three products, are indicated in the figures.

The increases observed in the anagen phase were:

group A: 16.8% at T₁ with respect to T₀;

group B: 22.2% at T₁ with respect to T₀;

group C, 7.65% at T₁ with respect to T₀;

In parallel, the telogen decreased in:

group A: 5.85% at T₁ with respect to T₀;

group B: 26.4% at T₁ with respect to T₀;

group C, 4.56% at T₁ with respect to T₀;

Haematochemical Analyses

There were no modifications in the haematochemical reference values in the subjects of group C, whereas a slight increase was noted in the proteins (albumin and alfa-1) in group A in 48% of the subjects, and a slight increase in the sideremia and ferritin, red blood cells and hemoglobin and serum-protein electrophoresis in 53% of the subjects of group B.

Pull Test

In group C the pull test score was not modified, whereas the pull resistance increased by 88.5% at T₁ in group A, and 89.4% at T₁ in group B.

Wash Test

Hair loss, objectively and subjectively evaluated by counting the number of hairs collected in the basin after washing (average of the subjective values in all the subjects for each washing), proved to be reduced, with respect to T₀ in:

group A: 57.2% at T₁;

group B: 65.7% at T₁;

group C, 0.5% at T₁;

Side-Effects

In group A, three subjects (5%) reported a mild heartburn after taking the capsule, this disturbance being solved by administration during the main meal.

In group B one subject reported increase in symptoms of spastic colitis with diarrhoea following administration of the capsule: this symptom spontaneously regressed with the fourteenth capsule and did not require suspension of the treatment.

In group C two subjects reported a mild heartburn after taking the capsule, probably due to the capsule shell. Also in this case, administration during the main meal solved the undesired symptom.

No other side-effect was reported during the experimentation.

Observations

In all types of alopecia, but above all in the telogenic effluvium form, maintenance of the anagen phase is the most suitable method for solving this form of trichological disease.

An ideal treatment for the cure of telogenic effluvium should therefore be aimed at controlling the cellular and biochemical homeostasis of the dermal papilla and other hair bulb structures, and attempting to neutralize (or better reduce) the various oxidative stimuli capable of triggering the transition from anagen to telogen of the hair bulb, by controlling the cellular apoptosis.

Very recent studies have shown how this process is also fundamental in various forms of androgenetic alopecia, thus revealing that the enzymatic mechanism of 5-α-reductase and aromatases is not the only one responsible for the pathology.

The results obtained from the double-blind study after administration of capsules containing extracts of Boehmeria extract alone and capsules containing Boehmeria extract and a pool of other trichogenic substances of Example 3, compared with the placebo, showed a synergic action of the various components.

In group A (capsules containing Boehmeria); a significant increase in the anagen value and a consequent decrease in the telogen value is observed. It is interesting to note that also the cathagen, obviously and contemporaneously tends to diminish due to an increase in the anagen phase.

There is consequently less hair loss (result of the wash test and pull test) and the diameter of the stem increases due to a recovery of the keratinisation of the dermal papilla.

No modification is observed in the main haematochemical values as the spermidine does not modify the synthesis of the haematic cells and does not produce oligoelements. There is however a modest increase in the proteins of the serum-protein electrophoresis.

In group B (formula of the food supplement of Example 3), the same modifications obtained with Boehmeria alone are obtained, with an increase in efficacy, probably due to the production of oligoelements, vitamins and antioxidants which generally improve the homeostasis of the hair synthesis.

In group C, there are no modifications in the symptomatology of telogenic effluvium. No objective or subjective result shows signs of improvement. This datum also demonstrates that the psychological component, in most of these forms, is not important.

It is also interesting to note that the diameter of the hair stem increases by 42.2% in the subjects of group A, and only by 49.8% in the subjects of group B. This result is extremely significant as it shows how Boehmeria alone is necessary and indispensable for the stimulation of the protein synthesis at the level of the cellular matrix, and consequently for the growth of the hair stems.

The other substances contained in the capsules administered to group B did not cause any significant modification of the synthesis of the stem.

The Wash test and Pull test are a more specific symptom of hair loss, and consequently of the progression of telogenic effluvium. In group A, at T₁, the number of hairs lost with washing decreased by 57.2%. In group B, at T₁, the number of hairs decreased by as much as 65.7%.

Consistently with the above evaluation, in this case all the other micronutrients provided with the final formulation of the new food supplement, improved the pathological situation of telogenic effluvium, confirming the fact that the oxidative action and supply of oligoelements and vitamins contributes to increasing the efficacy. Particularly, the pull resistance values of the hair stems improve more rapidly in group B with respect to group A.

The trichogram indicates the percentage variations in the hair cycle phases: according to the literature parameters, normal human trichogram values show about 79% of bulbs in anagen phase, 1% in cathagen phase and 20% in telogen phase.

New studies seem to indicate the presence of a further biological phase in the hair cycle, the exogen phase, following after telogen, which is the moment when the hair falls. This phase, morphologically different from the telogen phase, is the physiological phase of the detachment of the stem from the various anchorage systems to the derma, and its consequent falling. According to these increasingly accepted theories, falling in the telogen phase is a precocious detachment and consequently a pathological phase of hair loss.

The evaluation of the trichogram for this clinical study was done bearing in mind the different morphologies between telogen and exogen: only about 3.5% of the bulbs of all the samples could be classified as exogen at T₀.

An examination of the data shows that the capsules containing the active principles (group A and group B) were capable of increasing the number of bulbs in anagen phase and therefore reducing the telogen phase, with a consequent improvement in the clinical symptomatology.

A microscopic evaluation of a significant example of hair collected with the wash test at T₁ showed that in:

group A: 33% was in exogen phase (58% in telogen, 9% in cathagen);

group B: 46% was in exogen phase (51% in telogen, 3% in cathagen);

group C: only 3% was in exogen phase (81% in telogen, 16% in cathagen);

This latter result, not envisaged by the approved protocol as, at that moment, the exogen phase had not yet been described in standardized form, is extremely significant: the hair lost at the end of the study was in a different phase in the three groups, with a distinct predominance of the telogen phase in the placebo group, but with a significant number of bulbs in exogen (i.e. in a more “physiological” hair-falling phase) in the two groups which had taken the active products.

The lack of side-effects, definitely attributable to the administration of the products, leads to the conclusion that the capsules containing the active products are safe and have a low risk for undesired effects.

The double-blind clinical study for evaluating the efficacy of a food supplement, based on Boehmeria alone and Boehmeria associated with other nutritive principles, according to EXAMPLE 3 in the control of telogenic effluvium, compared with the placebo, showed that the administration of Boehmeria either alone or, above all; added to other active substances as in Example 3 (synergetic action), was capable of reducing the clinical symptoms and instrumental values relating to telogenic effluvium. The statistic difference in the data obtained with respect to the placebo group is significant as the placebo gave no modification in the clinical-instrumental symptoms.

From the study on healthy volunteers, no side-effects emerged, which could be attributed to the experimental products.

### Example 14

Determination in vivo of the inhibiting activity of 5-α-reductase of an extract of Boehmeria nippononivea.

The study was carried out comparing Boehmeria with Finasteride, which currently represents the most active inhibitor of the 5-α-reductase enzyme, responsible for the transformation of testosterone into the reduced, and more active, form: 5-α-dihydrotestosterone (DHT).

Materials and Methods

For the in-vivo study of the inhibition of 5-α-reductase, Sprague-Dawley (Charles River Italia) male adult rats were used having a body weight of 200-250 g.

The animals were stalled under standard conditions: at a temperature of 22/23° C., with 65% relative humidity, by exposing them to light cycles of 12 h light/12 h darkness.

A standard diet in pellets (standard diet, Charles River) was administered to the rats, together with water ad libitum.

The experiment was carried out according to protocols authorized by the committee for the care and use of animals of “Universita degli Studi di Milano”.

Samples were taken from the rear-orbital plexus, immediately before the pharmacological treatment (T₀) and then at 3, 6 and 8 hours after administration.

The administration of the substance being tested was effected orally.

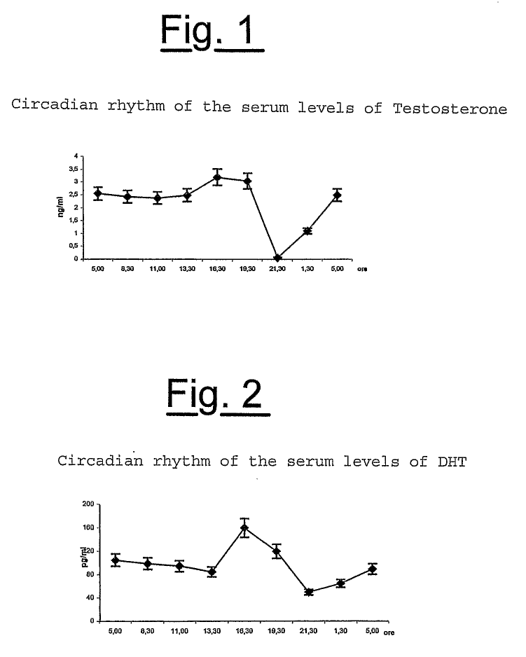

Furthermore, contemporarily with each sampling of the treated animals, samples were also taken from non-treated animals to determine the basal analyte level. Both the testosterone and DHT are in fact characterized by a significant circadian fluctuation as demonstrated in the enclosed FIGS. 1 and 2.

Plasma obtained from the whole blood, treated with EDTA after centrifugation, was preserved at −20° C. until analysis.

The plasmatic concentrations of DHT (dihydrotestosterone) were determined by a commercial kit (DSL, Chematil, Angri, SA) after extraction of the samples.

All the samples of an experimental set were analyzed together to reduce the inter analytical variability.

Results

The results are collected in the following table

| | | | Finasteride 1 mg | | | Finasteride 5 mg | | | Boehmeria 200 mg | | |
|---|---|---|---|---|---|---|---|---|---|---|---|
| Basal | | | 3 hrs | 6 hrs | 8 hrs | 3 hrs | 6 hrs | 8 hrs | 3 hrs | 6 hrs | 8 hrs |
| 345.38 | 397.11 | 720.75 | 298.09 | 404.13 | 706.70 | 217.10 | 103.62 | 201.56 | 285.67 | 107.86 | 140.17 |
| 266.38 | 162.70 | 350.44 | 505.28 | 565.00 | 140.06 | 618.58 | 54.35 | 108.76 | 185.59 | 89.17 | 67.03 |
| 474.16 | 272.95 | 824.51 | 300.00 | 379.59 | 503.90 | 151.18 | 60.67 | 745.40 | 384.41 | 118.89 | 47.80 |
| 290.82 | 195.61 | 615.65 | 569.62 | 592.06 | 530.07 | 640.97 | 49.67 | 576.60 | 793.18 | 85.82 | 56.43 |
| 89.83 | 138.12 | 62.50 | 404.63 | 148.51 | 173.11 | 924.20 | 128.25 | 250.90 | 154.00 | 138.78 | 94.47 |
| 191.77 | 434.36 | 114.50 | 530.00 | 420.07 | 110.00 | 569.38 | 176.92 | 300.12 | 107.65 | 376.63 | 167.67 |
| 337.07 | 673.64 | 46.37 | | | | | 81.23 | | | | |
| 88.55 | 344.97 | 905.26 | | | | | 205.21 | | | | |
| 102.65 | 581.58 | 63.72 | | | | | | | | | |
| 62.50 | 867.59 | 164.86 | | | | | | | | | |
| 91.88 | 506.37 | 1165.85 | | | | | | | | | |
| 171.52 | 206.16 | 389.53 | | | | | | | | | |
| 124.01 | 152.03 | 100.54 | | | | | | | | | |

| | | | | | | | | | | |
|---|---|---|---|---|---|---|---|---|---|---|
| n = | 39 | 6 | 6 | 6 | 6 | 8 | 6 | 6 | 6 | 6 |
| average | 202.809 | 434.603 | 418.227 | 360.640 | 520.235 | 107.490 | 363.890 | 318.42 | 152.86 | 95.60 |
| stand. errors | 35.71 | 48.30 | 64.86 | 102.57 | 118.03 | 20.64 | 99.85 | 103.27 | 45.46 | 19.85 |

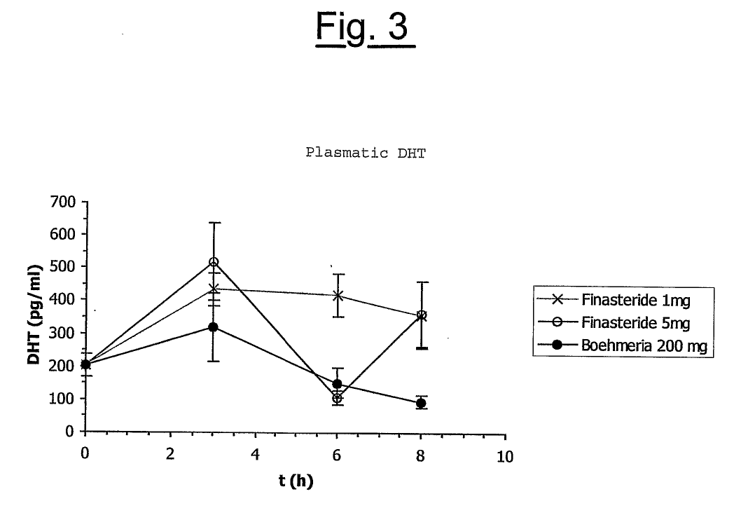

The graphic representation of the trend of the DHT concentration following administration, is shown in the enclosed FIG. 3.

The reductions in DHT concentrations are statistically significant: Boehmeria 8 h vs. basic p<0.04.

As can be seen from the data and the graph, the concentration of DHT is already reduced after the first three hours and reaches, at 6 hours, the same levels as that obtained with the administration of 5 mg of Finasteride.

The Boehmeria extract therefore shows an inhibiting capacity of 5-α-reductase quantitative comparable to that of Finasteride, but lasting longer. In fact with Finasteride the DHT, 8 hours after treatment, increases returning to the basal levels, whereas with Boehmeria the reduction is maintained at lower levels with statistic significance.

1. A composition for regulating the trophism of follicles and/or the skin production of sebum, comprising a synergic combination of
i) an extract of a vegetable origin which inhibits the 5-α-reductase enzyme, with
ii) at least one compound which acts at the level of the epithelial structures, characterized in that said extract of a vegetable origin
i) is an extract of a Boehmeria Nippononivea, and said compound acts at the level of the epithelial structures ii0 is an antioxidant compound.

2. The composition of claim 1, characterized in that said phenylpropanoids are selected from caffeic acid, hydroxytyrosole, chlorogenic acid, Teupolioside, Phenylpropanoids from Ajuga reptans, and mixtures thereof.

3. The composition according to claim 1, characterized in that said flavonoids are selected from flavonols, quercetin, Kaempferol, from isoflavones, genistein and daidzein, from catechine flavanols, or flavanones, nagenine and resveratrol and mixtures thereof.

4. The composition according to claim 1, characterized in that said isoprenoid derivatives are selected from carotenoids, tocopherols, tocotrienols, saponine and mixtures thereof.

5. The composition according to claim 1, characterized in that it is in a form for topic application.

6. Use of an extract of Boehmeria Nippononivea in association with an antioxidant compound selected from phenypropanoids, flavonoids, isoprenoid derivatives and mixtures thereof for the manufacture of a composition for the prevention or treatment of androgenetic alopecia and/or telogenic defluvium.

7. Use of an extract of Boehmeria Nippononivea in association with an antioxidant compound selected from phenypropanoids, flavonoids, isoprenoid derivatives and mixtures thereof for the manufacture of a composition in the prevention or treatment of acne and/or seborrhea.

8. Use of an extract of Boehmeria Nippononivea in association with an antioxidant compound selected from phenypropanoids, flavonoids, isoprenoid derivatives and mixtures thereof for the manufacture of a composition in the prevention or treatment of hypertricosis and/or hirsutism.

9. The composition according to claim 1 in a form suitable for oral administration.

10. Use of a food integrator comprising a composition according to claim 1, in the prevention or treatment of telogenic defluvium.

11. Use of a food integrator comprising a composition according to claim 1, in the prevention or treatment of androgenetic alopecia.

12. Use of a food integrator comprising a composition according to claim 1, in the prevention or treatment of acne and/or seborrhea.

13. Use of a food integrator comprising a composition according to claim 1, in the prevention or treatment of hypertricosis and/or hirsutism.
